(19) United States
(12) Patent Application Publication
(10) Pub. No.: US 2024/0016365 A1
(43) Pub. Date: Jan. 18, 2024

(54) IMAGE PROCESSING DEVICE, METHOD, AND PROGRAM

(71) Applicant: FUJIFILM Corporation, Tokyo (JP)

(72) Inventor: Sadato AKAHORI, Tokyo (JP)

(73) Assignee: FUJIFILM Corporation, Tokyo (JP)

(21) Appl. No.: 18/336,918

(22) Filed: Jun. 16, 2023

(30) Foreign Application Priority Data
Jul. 13, 2022 (JP) 2022-112631

(51) Int. Cl. A61B 1/00; G06T 19/00; G06T 7/70; A61B 6/12

(52) U.S. Cl. CPC A61B 1/00009; G06T 19/003; G06T 7/70; A61B 6/12; G06T 2210/41; G06T 2207/30064; G06T 2207/10068

(57) ABSTRACT

A processor acquires a three-dimensional image of a subject, acquires a radiation image of the subject having a lumen structure into which an endoscope is inserted, acquires a first real endoscopic image in the lumen structure of the subject captured at a first time point by the endoscope, derives a provisional virtual viewpoint in the three-dimensional image of the endoscope using the radiation image and the three-dimensional image, derives a virtual viewpoint at the first time point in the three-dimensional image of the endoscope using the provisional virtual viewpoint, the first real endoscopic image, and the three-dimensional image, and derives a virtual viewpoint at a second time point after the first time point in the three-dimensional image of the endoscope using the first real endoscopic image and a second real endoscopic image captured by the endoscope at the second time point.

### CROSS REFERENCE TO RELATED APPLICATIONS

The present application claims priority from Japanese Patent Application No. 2022-112631, filed on Jul. 13, 2022, the entire disclosure of which is incorporated herein by reference.

### BACKGROUND

### Technical Field

The present disclosure relates to an image processing device, method, and program.

### Related Art

An endoscope having an endoscopic observation part and an ultrasonic observation part at a distal end thereof is inserted into a lumen structure such as a digestive organ or a bronchus of a subject, and an endoscopic image in the lumen structure and an ultrasound image of a site such as a lesion located outside an outer wall of the lumen structure are captured. In addition, a biopsy in which a tissue of the lesion is collected with a treatment tool such as a forceps is also performed.

In a case of performing such a treatment using the endoscope, it is important that the endoscope accurately reaches a target position in the subject. Therefore, a positional relationship between the endoscope and a human body structure is grasped by continuously irradiating the subject with radiation from a radiation source during the treatment and performing fluoroscopic imaging to display the acquired fluoroscopic image in real time.

Here, since the fluoroscopic image includes overlapping anatomical structures such as organs, blood vessels, and bones in the subject, it is not easy to recognize the lumen and the lesion. Therefore, a three-dimensional image of the subject is acquired in advance before the treatment using a computed tomography (CT) device, a magnetic resonance imaging (MRI) device, and the like, an insertion route of the endoscope, a position of the lesion, and the like are simulated in advance in the three-dimensional image.

JP2009-056239A proposes a method of generating a virtual endoscopic image of an inside of a bronchus from a three-dimensional image, detecting a distal end position of an endoscope using a position sensor during a treatment, displaying the virtual endoscopic image together with a real endoscopic image captured by the endoscope, and performing insertion navigation of the endoscope into the bronchus.

In addition, JP2021-030073A proposes a method of detecting a distal end position of an endoscope with a position sensor provided at a distal end of the endoscope, detecting a posture of an imaging device that captures a fluoroscopic image using a lattice-shaped marker, reconstructing a three-dimensional image from a plurality of acquired fluoroscopic images, and performing registration between the reconstructed three-dimensional image and a three-dimensional image such as a CT image acquired in advance.

However, in the methods disclosed in JP2009-056239A and JP2021-030073A, it is necessary to provide a sensor in the endoscope in order to detect the position of the endoscope. In order to avoid using the sensor, detecting the position of the endoscope from an endoscopic image reflected in the fluoroscopic image is considered. However, since a position in a depth direction orthogonal to the fluoroscopic image is not known in the fluoroscopic image, a three-dimensional position of the endoscope cannot be detected from the fluoroscopic image. Therefore, it is not possible to perform accurate navigation of the endoscope to a desired position in the subject.

### SUMMARY OF THE INVENTION

The present invention has been made in view of the above circumstances, and an object of the present invention is to enable navigation of an endoscope to a desired position in a subject without using a sensor.

An image processing device according to a first aspect of the present disclosure comprises: at least one processor, in which the processor is configured to: acquire a three-dimensional image of a subject; acquire a radiation image of the subject having a lumen structure into which an endoscope is inserted; acquire a first real endoscopic image in the lumen structure of the subject captured at a first time point by the endoscope; derive a provisional virtual viewpoint in the three-dimensional image of the endoscope using the radiation image and the three-dimensional image; derive a virtual viewpoint at the first time point in the three-dimensional image of the endoscope using the provisional virtual viewpoint, the first real endoscopic image, and the three-dimensional image; and derive a virtual viewpoint at a second time point after the first time point in the three-dimensional image of the endoscope using the first real endoscopic image and a second real endoscopic image captured by the endoscope at the second time point.

A second aspect of the present disclosure provides the image processing device according to the first aspect of the present disclosure, in which the processor may be configured to: specify a position of the endoscope included in the radiation image; derive a position of the provisional virtual viewpoint using the specified position of the endoscope; and derive an orientation of the provisional virtual viewpoint using the position of the provisional virtual viewpoint in the three-dimensional image.

A third aspect of the present disclosure provides the image processing device according to the first or second aspect of the present disclosure, in which the processor may be configured to adjust the virtual viewpoint at the first time point such that a first virtual endoscopic image in the virtual viewpoint at the first time point derived using the three-dimensional image matches the first real endoscopic image. The term “match” includes not only a case of exact matching but also a case in which the positions are close to each other to the extent of substantial matching.

A fourth aspect of the present disclosure provides the image processing device according to any one of the first to third aspects of the present disclosure, in which the processor may be configured to: derive a change in viewpoint using the first real endoscopic image and the second real endoscopic image; and derive the virtual viewpoint at the second time point using the change in viewpoint and the virtual viewpoint at the first time point.

A fifth aspect of the present disclosure provides the image processing device according to the fourth aspect of the present disclosure, in which the processor may be configured to: determine whether or not an evaluation result representing a reliability degree with respect to the derived change in viewpoint satisfies a predetermined condition; and in a case in which the determination is negative, adjust the virtual viewpoint at the second time point such that a second virtual endoscopic image in the virtual viewpoint at the second time point matches the second real endoscopic image. The term “match” includes not only a case of exact matching but also a case in which the positions are close to each other to the extent of substantial matching.

A sixth aspect of the present disclosure provides the image processing device according to the fifth aspect of the present disclosure, in which the processor may be configured to, in a case in which the determination is affirmative, derive a third virtual viewpoint of the endoscope at a third time point after the second time point using the second real endoscopic image and a third real endoscopic image captured at the third time point.

A seventh aspect of the present disclosure provides the image processing device according to any one of the first to sixth aspects of the present disclosure, in which the processor may be configured to sequentially acquire a real endoscopic image at a new time point by the endoscope and sequentially derive a virtual viewpoint of the endoscope at each time point.

An eighth aspect of the present disclosure provides the image processing device according to the seventh aspect of the present disclosure, in which the processor may be configured to sequentially derive a virtual endoscopic image at each time point and sequentially display the real endoscopic image which is sequentially acquired and the virtual endoscopic image which is sequentially derived, using the three-dimensional image and the virtual viewpoint of the endoscope at each time point.

A ninth aspect of the present disclosure provides the image processing device according to the eighth aspect of the present disclosure, in which the processor may be configured to sequentially display the virtual endoscopic image at each time point and the real endoscopic image at each time point.

A tenth aspect of the present disclosure provides the image processing device according to the ninth aspect of the present disclosure, in which the processor may be configured to sequentially display a position of the virtual viewpoint at each time point in the lumen structure in the three-dimensional image.

An image processing method according to the present disclosure comprises: acquiring a three-dimensional image of a subject; acquiring a radiation image of the subject having a lumen structure into which an endoscope is inserted; acquiring a first real endoscopic image in the lumen structure of the subject captured at a first time point by the endoscope; deriving a provisional virtual viewpoint in the three-dimensional image of the endoscope using the radiation image and the three-dimensional image; deriving a virtual viewpoint at the first time point in the three-dimensional image of the endoscope using the provisional virtual viewpoint, the first real endoscopic image, and the three-dimensional image; and deriving a virtual viewpoint at a second time point after the first time point in the three-dimensional image of the endoscope using the first real endoscopic image and a second real endoscopic image captured by the endoscope at the second time point.

An image processing program according to the present disclosure causes a computer to execute a process comprising: acquiring a three-dimensional image of a subject; acquiring a radiation image of the subject having a lumen structure into which an endoscope is inserted; acquiring a first real endoscopic image in the lumen structure of the subject captured at a first time point by the endoscope; deriving a provisional virtual viewpoint in the three-dimensional image of the endoscope using the radiation image and the three-dimensional image; deriving a virtual viewpoint at the first time point in the three-dimensional image of the endoscope using the provisional virtual viewpoint, the first real endoscopic image, and the three-dimensional image; and deriving a virtual viewpoint at a second time point after the first time point in the three-dimensional image of the endoscope using the first real endoscopic image and a second real endoscopic image captured by the endoscope at the second time point.

According to the present disclosure, it is possible to perform navigation of an endoscope to a desired position in a subject without using a sensor.

### BRIEF DESCRIPTION OF THE DRAWINGS

FIG. 1 is a diagram showing a schematic configuration of a medical information system to which an image processing device according to an embodiment of the present disclosure is applied.

FIG. 2 is a diagram showing a schematic configuration of the image processing device according to the present embodiment.

FIG. 3 is a functional configuration diagram of the image processing device according to the present embodiment.

FIG. 4 is a diagram showing a fluoroscopic image.

FIG. 5 is a diagram for explaining derivation of a three-dimensional position of a viewpoint of a real endoscopic image.

FIG. 6 is a diagram for explaining a method of Shen et al.

FIG. 7 is a diagram for explaining a method of Zhou et al.

FIG. 8 is a diagram schematically showing processing performed by a second derivation unit.

FIG. 9 is a diagram for explaining derivation of an evaluation result representing a reliability degree of a change in viewpoint.

FIG. 10 is a diagram for explaining another example of the derivation of the evaluation result representing the reliability degree of the change in viewpoint.

FIG. 11 is a diagram showing a navigation screen.

FIG. 12 is a flowchart showing processing performed in the present embodiment.

FIG. 13 is a flowchart showing processing performed in the present embodiment.

### DETAILED DESCRIPTION

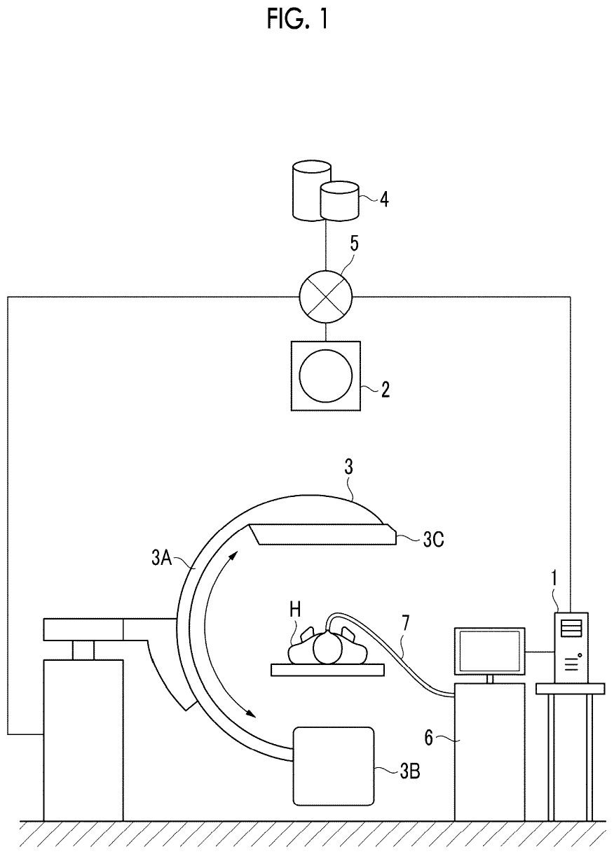

Hereinafter, embodiments of the present disclosure will be described with reference to the drawings. First, a configuration of a medical information system to which an image processing device according to the present embodiment is applied will be described. FIG. 1 is a diagram showing a schematic configuration of the medical information system. In the medical information system shown in FIG. 1, a computer 1 including the image processing device according to the present embodiment, a three-dimensional image capturing device 2, a fluoroscopic image capturing device 3, and an image storage server 4 are connected in a communicable state via a network 5.

The computer 1 includes the image processing device according to the present embodiment, and an image processing program of the present embodiment is installed in the computer 1. The computer 1 is installed in a treatment room where a subject is treated as described below. The computer 1 may be a workstation or a personal computer directly operated by a medical worker who performs a treatment or may be a server computer connected thereto via a network. The image processing program is stored in a storage device of the server computer connected to the network or in a network storage in a state of being accessible from the outside, and is downloaded and installed in the computer 1 used by a doctor in response to a request. Alternatively, the image processing program is distributed by being recorded on a recording medium such as a digital versatile disc (DVD) or a compact disc read only memory (CD-ROM) and is installed on the computer 1 from the recording medium.

The three-dimensional image capturing device 2 is a device that generates a three-dimensional image representing a treatment target site of a subject H by imaging the site, and is specifically, a CT device, an MRI device, a positron emission tomography (PET) device, and the like. The three-dimensional image including a plurality of tomographic images, which is generated by the three-dimensional image capturing device 2, is transmitted to and stored in the image storage server 4. In addition, in the present embodiment, the treatment target site of the subject H is a lung, and the three-dimensional image capturing device 2 is the CT device. A CT image including a chest portion of the subject H is acquired in advance as a three-dimensional image by imaging the chest portion of the subject H before a treatment on the subject H as described below and stored in the image storage server 4.

The fluoroscopic image capturing device 3 includes a C-arm 3A, an X-ray source 3B, and an X-ray detector 3C. The X-ray source 3B and the X-ray detector 3C are attached to both end parts of the C-arm 3A, respectively. In the fluoroscopic image capturing device 3, the C-arm 3A is configured to be rotatable and movable such that the subject H can be imaged from any direction. As will be described below, the fluoroscopic image capturing device 3 acquires an X-ray image of the subject H by performing fluoroscopic imaging in which the subject H is irradiated with X-rays during the treatment on the subject H, and the X-rays transmitted through the subject H are detected by the X-ray detector 3C. In the following description, the acquired X-ray image will be referred to as a fluoroscopic image. The fluoroscopic image is an example of a radiation image according to the present disclosure. A fluoroscopic image T0 may be acquired by continuously irradiating the subject H with X-rays at a predetermined frame rate, or by irradiating the subject H with X-rays at a predetermined timing such that an endoscope 7 reaches a branch of the bronchus as described below.

The image storage server 4 is a computer that stores and manages various types of data, and comprises a large-capacity external storage device and database management software. The image storage server 4 communicates with another device via the wired or wireless network 5 and transmits and receives image data and the like. Specifically, various types of data including image data of the three-dimensional image acquired by the three-dimensional image capturing device 2, and the fluoroscopic image acquired by the fluoroscopic image capturing device 3 are acquired via the network, and managed by being stored in a recording medium such as a large-capacity external storage device. A storage format of the image data and the communication between the respective devices via the network 5 are based on a protocol such as digital imaging and communication in medicine (DICOM).

In the present embodiment, it is assumed that a biopsy treatment is performed in which while performing fluoroscopic imaging of the subject H, a part of a lesion such as a pulmonary nodule existing in the lung of the subject H is excised to examine the presence or absence of a disease in detail. For this reason, the fluoroscopic image capturing device 3 is disposed in a treatment room for performing a biopsy. In addition, an ultrasonic endoscope device 6 is installed in the treatment room. The ultrasonic endoscope device 6 comprises an endoscope 7 whose distal end is attached with a treatment tool such as an ultrasound probe and a forceps. In the present embodiment, in order to perform a biopsy of the lesion, an operator inserts the endoscope 7 into the bronchus of the subject H, and captures a fluoroscopic image of the subject H with the fluoroscopic image capturing device 3 while capturing an endoscopic image of an inside of the bronchus by the endoscope 7. Then, the operator confirms a position of the endoscope 7 in the subject H in the fluoroscopic image while displaying the captured fluoroscopic image in real time, and moves a distal end of the endoscope 7 to a target position of the lesion. The bronchus is an example of the lumen structure of the present disclosure.

The endoscopic image is continuously acquired at a predetermined frame rate. In a case in which the fluoroscopic image T0 is acquired at a predetermined frame rate, a frame rate at which the endoscopic image is acquired may be the same as a frame rate at which the fluoroscopic image T0 is acquired. In addition, even in a case in which the fluoroscopic image T0 is acquired at an optional timing, the endoscopic image is acquired at a predetermined frame rate.

Here, lung lesions such as pulmonary nodules occur outside the bronchus rather than inside the bronchus. Therefore, after moving the endoscope 7 to the target position, the operator captures an ultrasound image of the outside of the bronchus with the ultrasound probe, displays the ultrasound image, and performs treatment of collecting a part of the lesion using a treatment tool such as a forceps while confirming a position of the lesion in the ultrasound image.

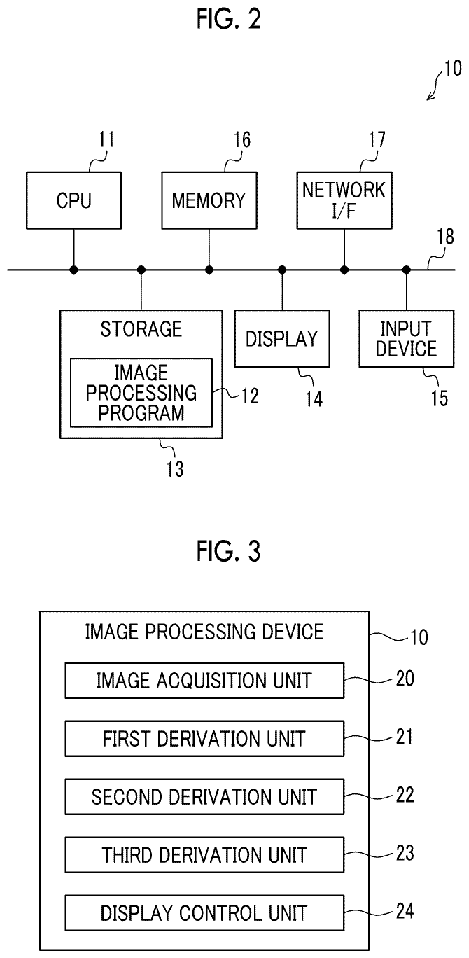

Next, the image processing device according to the present embodiment will be described. FIG. 2 is a diagram showing a hardware configuration of the image processing device according to the present embodiment. As shown in FIG. 2, the image processing device 10 includes a central processing unit (CPU) 11, a non-volatile storage 13, and a memory 16 as a temporary storage region. In addition, the image processing device 10 includes a display 14 such as a liquid crystal display, an input device 15 such as a keyboard and a mouse, and a network interface (UF) 17 connected to the network 5. The CPU 11, the storage 13, the display 14, the input device 15, the memory 16, and the network OF 17 are connected to a bus 18. The CPU 11 is an example of the processor in the present disclosure.

The storage 13 is realized by, for example, a hard disk drive (HDD), a solid state drive (SSD), a flash memory, and the like. An image processing program 12 is stored in the storage 13 as a storage medium. The CPU 11 reads out the image processing program 12 from the storage 13, expands the image processing program 12 in the memory 16, and executes the expanded image processing program 12.

Next, a functional configuration of the image processing device according to the present embodiment will be described. FIG. 3 is a diagram showing the functional configuration of the image processing device according to the present embodiment. As shown in FIG. 3, the image processing device 10 comprises an image acquisition unit 20, a first derivation unit 21, a second derivation unit 22, a third derivation unit 23, and a display control unit 24. Then, by executing the image processing program 12 by the CPU 11, the CPU 11 functions as the image acquisition unit 20, the first derivation unit 21, the second derivation unit 22, the third derivation unit 23, and the display control unit 24.

The image acquisition unit 20 acquires a three-dimensional image V0 of the subject H from the image storage server 4 in response to an instruction from the input device 15 by the operator. The acquired three-dimensional image V0 is assumed to be acquired before the treatment on the subject H. In addition, the image acquisition unit 20 acquires the fluoroscopic image T0 acquired by the fluoroscopic image capturing device 3 during the treatment of the subject H. Further, the image acquisition unit 20 acquires an endoscopic image R0 acquired by the endoscope 7 during the treatment of the subject H. The endoscopic image acquired by the endoscope 7 is acquired by actually imaging the inside of the bronchus of the subject H by the endoscope 7. Therefore, in the following description, the endoscopic image acquired by the endoscope 7 will be referred to as a real endoscopic image R0. The real endoscopic image R0 is acquired at a predetermined frame rate regardless of a method of acquiring the fluoroscopic image T0. Therefore, the real endoscopic image R0 is acquired at a timing close to a timing at which the fluoroscopic image T0 is acquired. Therefore, the real endoscopic image R0 whose acquisition timing corresponds to the acquisition timing of the fluoroscopic image T0 exists in the fluoroscopic image T0.

The first derivation unit 21 derives a provisional virtual viewpoint in the three-dimensional image V0 of the endoscope 7 using the fluoroscopic image T0 and the three-dimensional image V0. The first derivation unit 21, the second derivation unit 22, and the third derivation unit 23 may start processing using, for example, the fluoroscopic image T0 and the real endoscopic image R0 acquired after the distal end of the endoscope 7 reaches a first branch position of the bronchus, but the present invention is not limited to this. The processing may be performed after the insertion of the endoscope 7 into the subject H is started.

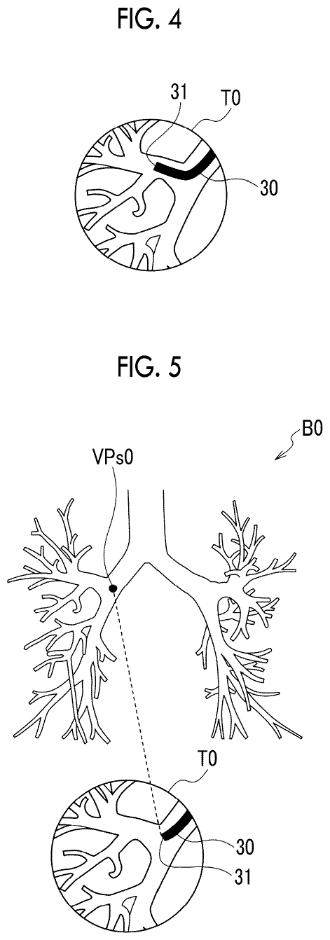

First, the first derivation unit 21 detects a position of the endoscope 7 from the fluoroscopic image T0. FIG. 4 is a diagram showing the fluoroscopic image. As shown in FIG. 4, the fluoroscopic image T0 includes an image 30 of the endoscope 7. The first derivation unit 21 uses, for example, a trained model trained to detect a distal end 31 of the endoscopic image 30 from the fluoroscopic image T0 to detect the distal end 31 of the endoscopic image 30 from the fluoroscopic image T0. The detection of the distal end 31 of the endoscopic image 30 from the fluoroscopic image T0 is not limited to this. Any method can be used, such as a method using template matching. The distal end 31 of the endoscopic image 30 detected in this manner serves as the position of the endoscope 7 in the fluoroscopic image T0.

Here, in the present embodiment, it is assumed that a bronchial region is extracted in advance from the three-dimensional image V0, the confirmation of the position of the lesion and the planning of a route to the lesion in the bronchus (that is, how and in that direction the endoscope 7 is inserted) are simulated in advance. The extraction of the bronchial region from the three-dimensional image V0 is performed using a known computer-aided diagnosis (CAD; hereinafter referred to as CAD) algorithm. In addition, for example, any method disclosed in JP2010-220742A is used.

In addition, the first derivation unit 21 performs registration between the fluoroscopic image T0 and the three-dimensional image V0. Here, the fluoroscopic image T0 is a two-dimensional image. Therefore, the first derivation unit 21 performs registration between the two-dimensional image and the three-dimensional image. In the present embodiment, first, the first derivation unit 21 projects the three-dimensional image V0 in the same direction as an imaging direction of the fluoroscopic image T0 to derive a two-dimensional pseudo fluoroscopic image VT0. Then, the first derivation unit 21 performs registration between the two-dimensional pseudo fluoroscopic image VT0 and the fluoroscopic image T0. As a method of the registration, any method such as rigid registration or non-rigid registration can be used.

On the other hand, since the fluoroscopic image T0 is two-dimensional, a position in the direction orthogonal to the fluoroscopic image T0, that is, a position in the depth direction is required in order to derive the provisional virtual viewpoint in the three-dimensional image V0. In the present embodiment, the bronchial region is extracted from the three-dimensional image V0 by the advance simulation. In addition, the first derivation unit 21 performs the registration between the fluoroscopic image T0 and the three-dimensional image V0. Therefore, as shown in FIG. 5, the distal end 31 of the endoscopic image 30 detected in the fluoroscopic image T0 is back-projected onto a bronchial region B0 of the three-dimensional image V0. Thereby, the position of the endoscope 7 in the three-dimensional image V0, that is, a three-dimensional position of a provisional virtual viewpoint VPs0 can be derived.

In addition, an insertion direction of the endoscope 7 into the bronchus is a direction from a mouth or nose toward an end of the bronchus. In a case in which the position in the bronchial region extracted from the three-dimensional image V0 is known, the direction of the endoscope 7 at that position, that is, the direction of the provisional virtual viewpoint VPs0 is known. In addition, a method of inserting the endoscope 7 into the subject H is predetermined. For example, at a start of the insertion of the endoscope 7, a method of inserting the endoscope 7 is predetermined such that a ventral side of the subject H is an upper side of the real endoscopic image. Therefore, a degree to which the endoscope 7 is twisted around its major axis in the position of the derived viewpoint can be derived by the above-described advance simulation based on a shape of the bronchial region. Therefore, the first derivation unit 21 derives a degree of twist of the endoscope 7 at the derived position of the provisional virtual viewpoint using a result of the simulation. Thereby, the first derivation unit 21 derives an orientation of the provisional virtual viewpoint VPs0 in the three-dimensional image V0. In the present embodiment, deriving the provisional virtual viewpoint VPs0 means deriving a three-dimensional position and an orientation (that is, the line-of-sight direction and the twist) of the viewpoint in the three-dimensional image V0 of the provisional virtual viewpoint VPs0.

The second derivation unit 22 uses the provisional virtual viewpoint VPs0 derived by the first derivation unit 21, a real endoscopic image R1 at a first time point t1, and the three-dimensional image V0 to derive a first virtual viewpoint VP1 at the first time point t1 in the three-dimensional image V0 of the endoscope 7. In the present embodiment, the second derivation unit 22 derives the first virtual viewpoint VP1 using a method disclosed in “Context-Aware Depth and Pose Estimation for Bronchoscopic Navigation IEEE Robotics and Automation Letters, Mali Shen et al., Vol. 4, no. 2, pp. 732 to 739, April 2019”. Here, the real endoscopic image R0 is continuously acquired at a predetermined frame rate, but in the present embodiment, the real endoscopic image R0 acquired at the first time point t1, which is conveniently set for processing in the second derivation unit 22, is set as the first real endoscopic image R1.

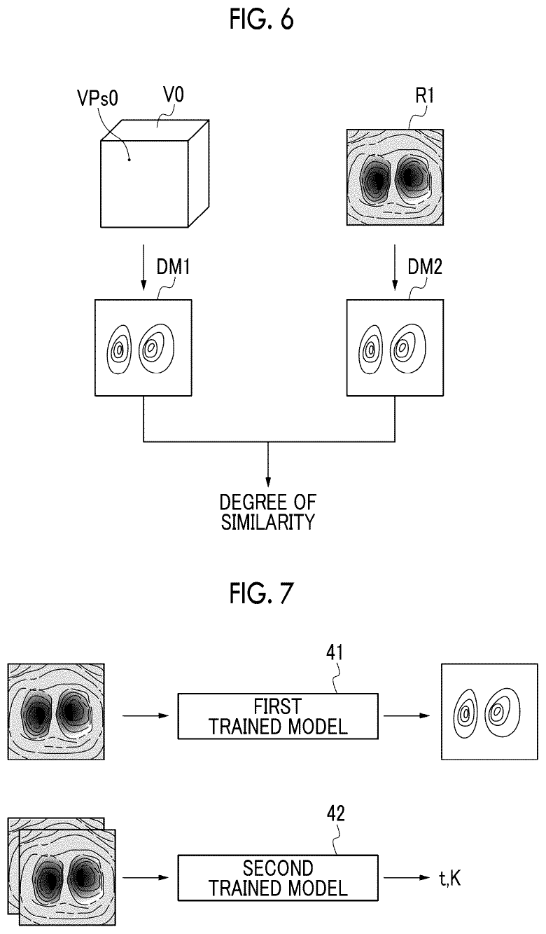

FIG. 6 is a diagram illustrating adjustment of a virtual viewpoint using a method of Shen et al. As shown in FIG. 6, the second derivation unit 22 analyzes the three-dimensional image V0 to derive a depth map (referred to as a first depth map) DM1 in a traveling direction of the endoscope 7 at the provisional virtual viewpoint VPs0. Specifically, the first depth map DM1 at the provisional virtual viewpoint VPs0 is derived using the bronchial region extracted in advance from the three-dimensional image V0 as described above. In FIG. 6, only the provisional virtual viewpoint VPs0 in the three-dimensional image V0 is shown, and the bronchial region is omitted. In addition, the second derivation unit 22 derives a depth map (referred to as a second depth map) DM2 of the first real endoscopic image R1 by analyzing the first real endoscopic image R1. The depth map is an image in which a depth of an object in a direction in which the viewpoint is directed is represented by a pixel value, and represents a distribution of a distance in the depth direction in the image.

For the derivation of the second depth map DM2, for example, a method disclosed in “Unsupervised Learning of Depth and Ego-Motion from Video, Tinghui Zhou et al., April 2017” can be used. FIG. 7 is a diagram illustrating the method of Zhou et al. As shown in FIG. 7, the document of Zhou et al. discloses a method of training a first trained model 41 for deriving a depth map and a second trained model 42 for deriving a change in line of sight. The second derivation unit 22 derives a depth map using the first trained model 41 trained by the method disclosed in the document of Zhou et al. The first trained model 41 is constructed by subjecting a neural network to machine learning such that a depth map representing a distribution of a distance in a depth direction of one frame constituting a video image is derived from the frame.

The second trained model 42 is constructed by subjecting a neural network to machine learning such that a change in viewpoint between two frames constituting a video image is derived from the two frames. The change in viewpoint is a parallel movement amount t of the viewpoint and an amount of change in orientation between frames, that is, a rotation amount K.

In the method of Zhou et al., the first trained model 41 and the second trained model 42 are simultaneously trained without using training data, based on a relational expression between the change in viewpoint and the depth map to be satisfied between a plurality of frames. The first trained model 41 may be constructed using a large number of learning data including an image for training and a depth map as correct answer data for the image for training, without using the method of Zhou et al. In addition, the second trained model 42 may be constructed using a large number of learning data including a combination of two images for training and changes in viewpoints of the two images which are correct answer data.

Then, while changing the provisional virtual viewpoint VPs0, the second derivation unit 22 derives the first depth map DM1 in the changed provisional virtual viewpoint VPs0. In addition, the second derivation unit 22 derives the second depth map DM2 from the first real endoscopic image R1 at the first time point t1. Subsequently, the second derivation unit 22 derives a degree of similarity between the first depth map DM1 and the second depth map DM2. Then, the provisional virtual viewpoint VPs0 having the maximum degree of similarity is derived as the first virtual viewpoint VP1 at the first time point t1.

The third derivation unit 23 uses a second real endoscopic image R2 captured by the endoscope 7 at a second time point t2 after the first time point t1 and the first real endoscopic image R1 acquired at the first time point t1 to derive a second virtual viewpoint VP2 at the second time point t2 in the three-dimensional image V0 of the endoscope 7.

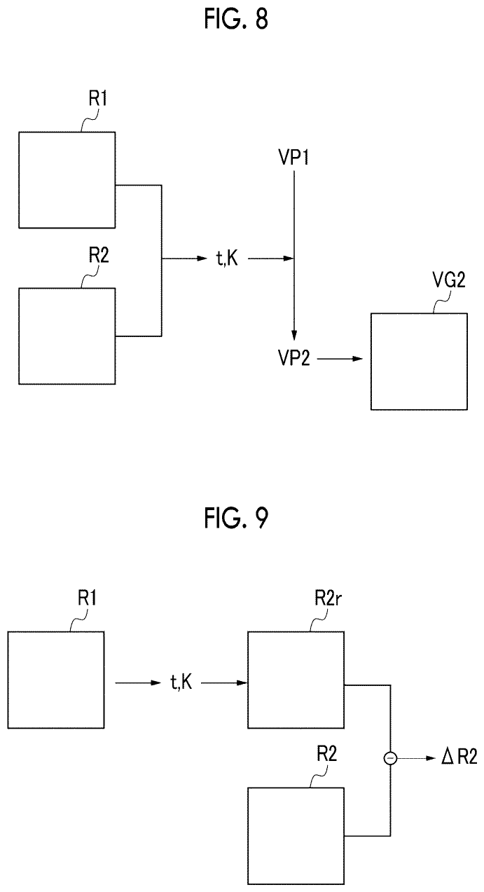

FIG. 8 is a diagram schematically showing processing performed by the third derivation unit 23. As shown in FIG. 8, first, the third derivation unit 23 uses the second trained model 42 disclosed in the above-described document of Zhou et al. to derive a change in viewpoint from the first real endoscopic image R1 to the second real endoscopic image R2. It is also possible to derive a change in viewpoint from the second real endoscopic image R2 to the first real endoscopic image R1 by changing an input order of the first real endoscopic image R1 and the second real endoscopic image R2 of the image to the second trained model 42. The change in viewpoint is derived as the parallel movement amount t and the rotation amount K of the viewpoint from the first real endoscopic image R1 to the second real endoscopic image R2.

Then, the third derivation unit 23 derives the second virtual viewpoint VP2 by converting the first virtual viewpoint VP1 derived by the second derivation unit 22 using the derived change in viewpoint. Further, the third derivation unit 23 derives a second virtual endoscopic image VG2 in the second virtual viewpoint VP2. For example, the third derivation unit 23 derives the virtual endoscopic image VG2 by using a method disclosed in JP2020-010735A. Specifically, a projection image is generated by performing central projection in which the three-dimensional image V0 on a plurality of lines of sight radially extending in a line-of-sight direction of the endoscope from the second virtual viewpoint VP2 is projected onto a predetermined projection plane. This projection image is the virtual endoscopic image VG2 that is virtually generated as though the image has been captured at the distal end position of the endoscope. As a specific method of central projection, for example, a known volume rendering method or the like can be used.

In the present embodiment, the image acquisition unit 20 sequentially acquires the real endoscopic image R0 captured by the endoscope 7 at a predetermined frame rate. The third derivation unit 23 uses the latest real endoscopic image R0 as the second real endoscopic image R2 at the second time point t2 and the real endoscopic image R0 acquired one time point before the second time point t2 as the first real endoscopic image R1 at the first time point t1, to derive the second virtual viewpoint VP2 at a time point at which the second real endoscopic image R2 is acquired. The derived second virtual viewpoint VP2 is the viewpoint of the endoscope 7. In addition, the third derivation unit 23 sequentially derives the virtual endoscopic image VG2 in the second virtual viewpoint VP2 that is sequentially derived.

Here, in a case in which the endoscope 7 moves relatively slowly in the bronchus, the change in viewpoint by the third derivation unit 23 can be derived with a relatively high accuracy. On the other hand, in a case in which the endoscope 7 moves rapidly in the bronchus, the derivation accuracy of the change in viewpoint by the third derivation unit 23 may decrease. Therefore, in the present embodiment, the third derivation unit 23 derives an evaluation result representing a reliability degree of the derived change in viewpoint, and determines whether or not the evaluation result satisfies the predetermined condition.

FIG. 9 is a diagram for explaining the derivation of the evaluation result representing the reliability degree of the change in viewpoint. In deriving the evaluation result representing the reliability degree, the third derivation unit 23 uses the change in viewpoint from the first real endoscopic image R1 to the second real endoscopic image R2 (that is, t and K) derived as described above, to convert the real endoscopic image R1 at the first time point t1, thereby deriving a converted real endoscopic image R2r whose viewpoint is converted. The converted real endoscopic image R2r corresponds to the second real endoscopic image R2 at the second time point t2. Then, the third derivation unit 23 derives a difference ΔR2 between the second real endoscopic image R2 at the second time point t2 and the converted real endoscopic image R2r as the evaluation result representing the reliability degree of the change in viewpoint. As the difference ΔR2, a sum of absolute values of difference values between pixel values of corresponding pixels of the second real endoscopic image R2 and the converted real endoscopic image R2r, or a sum of squares of the difference values can be used.

Here, in a case in which the change in viewpoint between the first real endoscopic image R1 and the second real endoscopic image R2 is derived with a high accuracy, the converted real endoscopic image R2r matches the second real endoscopic image R2, so that the difference ΔR2 decreases. On the other hand, in a case in which the derivation accuracy of the change in viewpoint between the first real endoscopic image R1 and the second real endoscopic image R2 is low, the converted real endoscopic image R2r does not match the second real endoscopic image R2, so that the difference ΔR2 increases. Therefore, the smaller the difference ΔR2, which is the evaluation result representing the reliability degree, the higher the reliability degree of the change in viewpoint.

Therefore, the third derivation unit 23 determines whether or not the evaluation result representing the reliability degree with respect to the change in viewpoint satisfies the predetermined condition, based on whether or not the difference ΔR2 is smaller than a predetermined threshold value Th1.

In a case in which the determination is negative, the third derivation unit 23 adjusts the second virtual viewpoint VP2 such that the second virtual endoscopic image VG2 in the virtual viewpoint VP2 at the second time point t2 matches the second real endoscopic image R2. The adjustment of the second virtual viewpoint VP2 is performed by using the method of Shen et al. described above. That is, the third derivation unit 23 derives a depth map DM3 using the bronchial region extracted from the three-dimensional image V0 while changing the virtual viewpoint VP2, and derives a degree of similarity between the depth map DM3 and the depth map DM2 of the second real endoscopic image R2. Then, the virtual viewpoint VP2 having the maximum degree of similarity is determined as a new virtual viewpoint VP2 at the second time point t2.

The third derivation unit 23 uses the new virtual viewpoint VP2 to derive a new change in viewpoint from the virtual viewpoint VP1 to the new virtual viewpoint VP2. The third derivation unit 23 derives a new converted real endoscopic image R2r by converting the real endoscopic image R1 at the first time point t1 using the new change in viewpoint. Then, the third derivation unit 23 derives a new difference ΔR2 between the second real endoscopic image R2 at the second time point t2 and the new converted real endoscopic image R2r as an evaluation result representing a new reliability degree, and determines again whether or not the evaluation result representing the new reliability degree satisfies the above predetermined condition.

In a case in which the second determination is negative, the image acquisition unit 20 acquires a new fluoroscopic image T0, and the derivation of the provisional virtual viewpoint by the first derivation unit 21, the derivation of the virtual viewpoint VP1 at the first time point t1 by the second derivation unit 22, and the derivation of the virtual viewpoint VP2 at the second time point t2 by the third derivation unit 23 are performed again.

In a case in which the first determination is affirmative, or in a case in which the second determination is affirmative after the first determination is negative, the third derivation unit 23 updates a third real endoscopic image R3 acquired at a time point after the second time point t2 (referred to as a third time point t3) and the second real endoscopic image R2 to the second real endoscopic image R2 and the first real endoscopic image R1, respectively, to derive a virtual viewpoint VP3 at the third time point t3, that is, the updated second virtual viewpoint VP2 at the second time point t2. By repeating this process for the real endoscopic image R0 that is continuously acquired, the virtual viewpoint VP0 of the endoscope 7 is sequentially derived, and a virtual endoscopic image VG0 in the virtual viewpoint VP0 that is sequentially derived is sequentially derived.

In addition, the third derivation unit 23 may determine the reliability degree of the change in viewpoint only once, and, in a case in which the determination is negative, the processing of the first derivation unit 21, the second derivation unit 22 and the third derivation unit 23 may be performed using the new fluoroscopic image T0 without adjusting the new virtual viewpoint VP2.

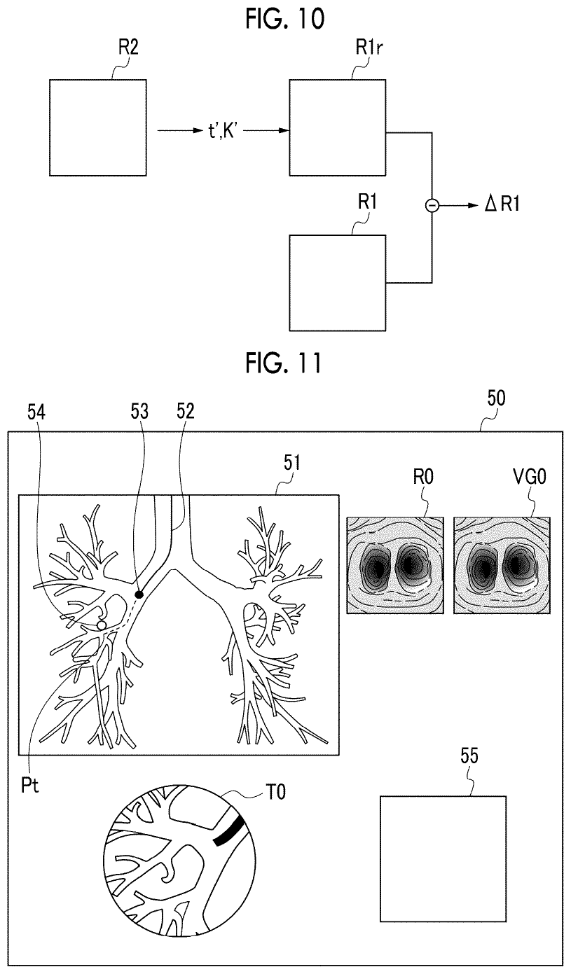

On the other hand, the third derivation unit 23 may derive the evaluation result representing the reliability degree of the change in viewpoint as follows. FIG. 10 is a diagram for explaining another derivation of the reliability degree of the change in viewpoint. The third derivation unit 23 first derives the difference ΔR2 using the second trained model 42 in the same manner as described above. In addition, the third derivation unit 23 derives the change in viewpoint from the second real endoscopic image R2 to the first real endoscopic image R1 by changing the input order of the image to the second trained model 42. This change in viewpoint is derived as t′ and K′. The third derivation unit 23 derives a converted real endoscopic image R1r whose viewpoint is converted by converting the second real endoscopic image R2 at the second time point t2 using the change in viewpoint, that is, t′ and K′. The converted real endoscopic image R1r corresponds to the first real endoscopic image R1 at the first time point t1. Then, a difference ΔR1 between the first real endoscopic image R1 at the first time point t1 and the converted real endoscopic image R1r is derived. As the difference ΔR1, a sum of absolute values of difference values between pixel values of corresponding pixels of the first real endoscopic image R1 and the converted real endoscopic image R1r, or a sum of squares of the difference values can be used.

Then, the third derivation unit 23 derives an evaluation result representing the reliability degree of the change in viewpoint using both the difference ΔR2 and the difference ΔR1. In this case, as the evaluation result, a representative value between the difference ΔR2 and the difference ΔR1, such as an average of the difference ΔR2 and the difference ΔR1 or a smaller difference between the difference ΔR2 and the difference ΔR1, can be used. Then, the third derivation unit 23 determines whether or not the derived evaluation result satisfies the predetermined condition, and performs the same processing as described above according to a result of the determination.

The display control unit 24 displays a navigation screen including the fluoroscopic image T0, the real endoscopic image R0, and the virtual endoscopic image VG0 on the display 14. In addition, as necessary, an ultrasound image acquired by the ultrasonic endoscope device 6 is included in the navigation screen and displayed. FIG. 11 is a diagram showing the navigation screen. As shown in FIG. 11, an image 51 of the bronchial region included in the three-dimensional image V0, the fluoroscopic image T0, the real endoscopic image R0, and the virtual endoscopic image VG0 are displayed on the navigation screen 50. The real endoscopic image R0 is an image acquired by the endoscope 7 at a predetermined frame rate, and the virtual endoscopic image VG0 is an image derived corresponding to the real endoscopic image R0. The fluoroscopic image T0 is an image acquired at a predetermined frame rate or a predetermined timing.

On the navigation screen 50, the image 51 of the bronchial region displays a route 52 for navigation of the endoscope 7 to a target point Pt where a lesion 54 exists. In addition, a current position 53 of the endoscope 7 is shown on the route 52. The position 53 corresponds to the latest virtual viewpoint VP0 derived by the third derivation unit 23. The displayed real endoscopic image R0 and virtual endoscopic image VG0 are a real endoscopic image and a virtual endoscopic image at the position 53.

In addition, in FIG. 11, the route 52 through which the endoscope 7 has passed is shown by a solid line, and the route 52 through which the endoscope 7 has not passed is shown by a broken line. The navigation screen 50 has a display region 55 for an ultrasound image, and the ultrasound image acquired by the ultrasonic endoscope device 6 is displayed in the display region 55.

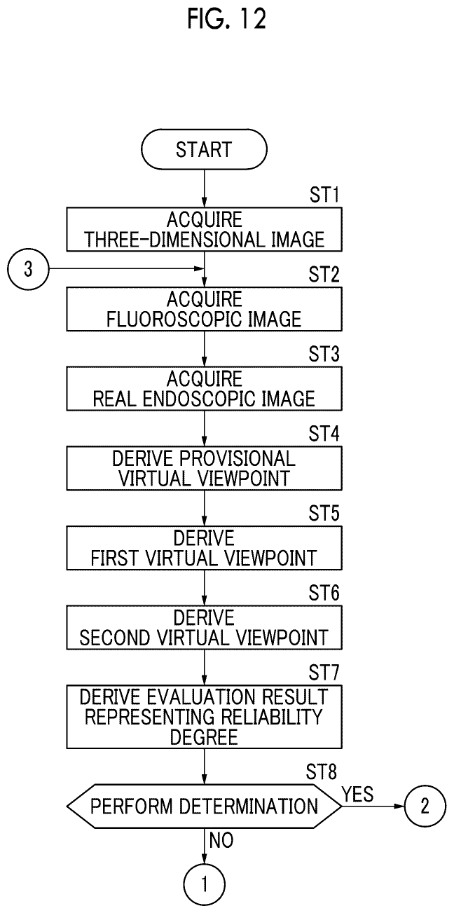

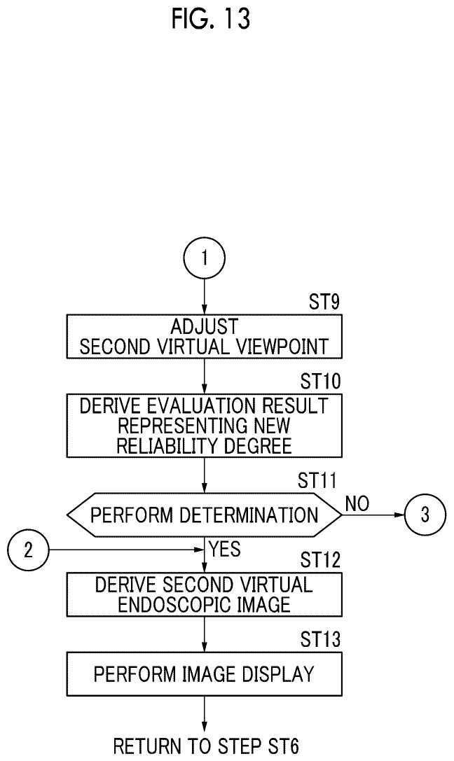

Next, processing performed in the present embodiment will be described. FIGS. 12 and 13 are a flowchart showing the processing performed in the present embodiment. First, the image acquisition unit 20 acquires the three-dimensional image V0 from the image storage server 4 (step ST1), acquires the fluoroscopic image T0 (step ST2), and further acquires the real endoscopic image R0 (step ST3). The real endoscopic image acquired in step ST3 is the first real endoscopic image R1 at the first time point t1 and the second real endoscopic image R2 at the second time point t2, of which the acquisition time points are adjacent to each other at a start of the processing. This is a real endoscopic image with the latest imaging time point after the processing is started.

Next, the first derivation unit 21 derives the provisional virtual viewpoint VPs0 in the three-dimensional image V0 of the endoscope 7 using the fluoroscopic image T0 and the three-dimensional image V0 (step ST4). Subsequently, the second derivation unit 22 uses the provisional virtual viewpoint VPs0 derived by the first derivation unit 21, the first real endoscopic image R1, and the three-dimensional image V0 to derive the first virtual viewpoint VP1 at the first time point t1 in the three-dimensional image V0 of the endoscope 7 (step ST5).

Next, the third derivation unit 23 uses the second real endoscopic image R2 captured by the endoscope 7 at the second time point t2 after the first time point t1 and the first real endoscopic image R1 acquired at the first time point t1 to derive the second virtual viewpoint VP2 at the second time point t2 in the three-dimensional image V0 of the endoscope 7 (step ST6).

Subsequently, the third derivation unit 23 derives an evaluation result representing the reliability degree of the change in viewpoint (step ST7), and determines whether or not the evaluation result representing the reliability degree with respect to the change in viewpoint satisfies the predetermined condition (step ST8). In a case in which step ST8 is negative, the third derivation unit 23 adjusts the second virtual viewpoint VP2 (step ST9), and derives an evaluation result representing a new reliability degree using the adjusted new virtual viewpoint VP2 (Step ST10). Then, it is determined whether or not the evaluation result representing the new reliability degree satisfies the predetermined condition (step ST11). In a case in which step ST11 is negative, the process returns to step ST2, a new fluoroscopic image T0 is acquired, and the process after step ST2 using the new fluoroscopic image T0 is repeated.

In a case in which steps ST8 and ST11 are affirmative, the third derivation unit 23 derives the second virtual endoscopic image VG2 in the latest second virtual viewpoint VP2 (step ST12). Then, the display control unit 24 displays the navigation screen including the image 51 of the bronchial region, the real endoscopic image R0, and the virtual endoscopic image VG0 on the display 14 (image display: step ST13). The real endoscopic image R0 displayed at this time point is the latest second real endoscopic image R2, and the virtual endoscopic image VG0 is the second virtual endoscopic image VG2 corresponding to the latest second real endoscopic image R2. The first real endoscopic image R1 and the first virtual endoscopic image VG1 may be displayed before these displays. Then, the process returns to step ST6, and the process after step ST6 is repeated. Thereby, the real endoscopic image R0 which is sequentially acquired and the virtual endoscopic image VG0 in the viewpoint registered with the viewpoint of the real endoscopic image R0 are displayed on the navigation screen 50.

As described above, in the present embodiment, the provisional virtual viewpoint VPs0 in the three-dimensional image V0 of the endoscope 7 is derived using the fluoroscopic image TO and the three-dimensional image V0, the virtual viewpoint VP1 at the first time point t1 in the three-dimensional image V0 of the endoscope 7 is derived using the provisional virtual viewpoint VPs0, the first real endoscopic image R1, and the three-dimensional image V0, and the virtual viewpoint VP2 at the second time point t2 in the three-dimensional image V0 of the endoscope 7 is derived using the second real endoscopic image R2 and the first real endoscopic image R1. Therefore, even though the distal end of the endoscope 7 is not detected using the sensor, by deriving the virtual endoscopic image VG2 in the virtual viewpoint VP2, navigation of the endoscope 7 to a desired position in the subject H can be performed using the derived virtual endoscopic image VG2.

In addition, by adjusting the virtual viewpoint VP1 at the first time point t1 such that the first virtual endoscopic image VG1 in the virtual viewpoint VP1 at the first time point t1 matches the first real endoscopic image R1, the virtual endoscopic image VG1 and even the virtual endoscopic image VG2 of the viewpoint matching the actual viewpoint of the endoscope 7 can be derived.

In addition, by deriving a change in viewpoint using the first real endoscopic image R1 and the second real endoscopic image R2 and the virtual viewpoint VP2 at the second time point t2 using the change in viewpoint and the virtual viewpoint VP1 at the first time point t1, a new virtual viewpoint VP2 can be derived with a high accuracy. Therefore, it is possible to derive the virtual endoscopic image VG2 of the viewpoint matching the actual viewpoint of the endoscope 7.

In addition, it is determined whether or not an evaluation result representing the reliability degree with respect to the change in viewpoint satisfies a predetermined condition using the first real endoscopic image R1 and the second real endoscopic image R2, and, in a case in which the determination is negative, the virtual viewpoint VP2 at the second time point t2 is adjusted, thereby deriving the virtual viewpoint VP2 with a high accuracy, and as a result, it is possible to derive the virtual endoscopic image VG2 of the viewpoint matching the actual viewpoint of the endoscope 7.

In this case, it is further determined whether or not an evaluation result representing a reliability degree of a new change in viewpoint based on the adjusted virtual viewpoint VP2 satisfies the predetermined condition, and, in a case in which the further determination is negative, a new fluoroscopic image T0 is acquired, and the processing of the first derivation unit 21, the second derivation unit 22 and the third derivation unit 23 is performed again, whereby the deviation between the position of the endoscope 7 and the virtual viewpoint VP2 over time can be corrected. Therefore, the virtual viewpoint VP2 can be derived with a high accuracy.

In the above-described embodiment, a case in which the image processing device of the present disclosure is applied to observation of the bronchus has been described, but the present disclosure is not limited thereto, and the present disclosure can also be applied in a case in which a lumen structure such as a stomach, a large intestine, and a blood vessel is observed with an endoscope.

In addition, in the above-described embodiment, for example, as a hardware structure of a processing unit that executes various types of processing such as the image acquisition unit 20, the first derivation unit 21, the second derivation unit 22, the third derivation unit 23, and the display control unit 24, various processors shown below can be used. The various types of processors include, as described above, a CPU which is a general-purpose processor that executes software (program) to function as various types of processing units, as well as a programmable logic device (PLD) which is a processor having a circuit configuration that can be changed after manufacturing such as a field programmable gate array (FPGA), a dedicated electrical circuit which is a processor having a circuit configuration exclusively designed to execute specific processing such as an application specific integrated circuit (ASIC), and the like.

One processing unit may be configured of one of the various types of processors, or a combination of two or more processors of the same type or different types (for example, a combination of a plurality of FPGAs, or a combination of a CPU and an FPGA). Further, a plurality of processing units may be configured of one processor.

As an example of configuring a plurality of processing units with one processor, first, there is a form in which, as typified by computers such as a client and a server, one processor is configured by combining one or more CPUs and software, and the processor functions as a plurality of processing units. Second, there is a form in which, as typified by a system on chip (SoC) and the like, in which a processor that implements functions of an entire system including a plurality of processing units with one integrated circuit (IC) chip is used. As described above, the various types of processing units are configured using one or more of the various types of processors as a hardware structure.

Furthermore, as the hardware structure of the various types of processors, more specifically, an electric circuit (circuitry) in which circuit elements such as semiconductor elements are combined can be used.

What is claimed is:

1. An image processing device comprising:
at least one processor,
wherein the processor is configured to:
acquire a three-dimensional image of a subject;
acquire a radiation image of the subject having a lumen structure into which an endoscope is inserted;
acquire a first real endoscopic image in the lumen structure of the subject captured at a first time point by the endoscope;
derive a provisional virtual viewpoint in the three-dimensional image of the endoscope using the radiation image and the three-dimensional image;
derive a virtual viewpoint at the first time point in the three-dimensional image of the endoscope using the provisional virtual viewpoint, the first real endoscopic image, and the three-dimensional image; and
derive a virtual viewpoint at a second time point after the first time point in the three-dimensional image of the endoscope using the first real endoscopic image and a second real endoscopic image captured by the endoscope at the second time point.

2. The image processing device according to claim 1,
wherein the processor is configured to:
specify a position of the endoscope included in the radiation image;
derive a position of the provisional virtual viewpoint using the specified position of the endoscope; and
derive an orientation of the provisional virtual viewpoint using the position of the provisional virtual viewpoint in the three-dimensional image.

3. The image processing device according to claim 1,
wherein the processor is configured to adjust the virtual viewpoint at the first time point such that a first virtual endoscopic image in the virtual viewpoint at the first time point derived using the three-dimensional image matches the first real endoscopic image.

4. The image processing device according to claim 1,
wherein the processor is configured to:
derive a change in viewpoint using the first real endoscopic image and the second real endoscopic image; and
derive the virtual viewpoint at the second time point using the change in viewpoint and the virtual viewpoint at the first time point.

5. The image processing device according to claim 4,
wherein the processor is configured to:
determine whether or not an evaluation result representing a reliability degree with respect to the derived change in viewpoint satisfies a predetermined condition; and
in a case in which the determination is negative, adjust the virtual viewpoint at the second time point such that a second virtual endoscopic image in the virtual viewpoint at the second time point matches the second real endoscopic image.

6. The image processing device according to claim 5,
wherein the processor is configured to, in a case in which the determination is affirmative, derive a third virtual viewpoint of the endoscope at a third time point after the second time point using the second real endoscopic image and a third real endoscopic image captured at the third time point.

7. The image processing device according to claim 1,
wherein the processor is configured to sequentially acquire a real endoscopic image at a new time point by the endoscope and sequentially derive a virtual viewpoint of the endoscope at each time point.

8. The image processing device according to claim 7,
wherein the processor is configured to sequentially derive a virtual endoscopic image at each time point and sequentially display the real endoscopic image which is sequentially acquired and the virtual endoscopic image which is sequentially derived, using the three-dimensional image and the virtual viewpoint of the endoscope at each time point.

9. The image processing device according to claim 8,
wherein the processor is configured to sequentially display the virtual endoscopic image at each time point and the real endoscopic image at each time point.

10. The image processing device according to claim 9,
wherein the processor is configured to sequentially display a position of the virtual viewpoint at each time point in the lumen structure in the three-dimensional image.

11. An image processing method comprising:
acquiring a three-dimensional image of a subject;
acquiring a radiation image of the subject having a lumen structure into which an endoscope is inserted;
acquiring a first real endoscopic image in the lumen structure of the subject captured at a first time point by the endoscope;
deriving a provisional virtual viewpoint in the three-dimensional image of the endoscope using the radiation image and the three-dimensional image;
deriving a virtual viewpoint at the first time point in the three-dimensional image of the endoscope using the provisional virtual viewpoint, the first real endoscopic image, and the three-dimensional image; and
deriving a virtual viewpoint at a second time point after the first time point in the three-dimensional image of the endoscope using the first real endoscopic image and a second real endoscopic image captured by the endoscope at the second time point.

12. A non-transitory computer-readable storage medium that stores an image processing program causing a computer to execute a process comprising:
acquiring a three-dimensional image of a subject;
acquiring a radiation image of the subject having a lumen structure into which an endoscope is inserted;
acquiring a first real endoscopic image in the lumen structure of the subject captured at a first time point by the endoscope;
deriving a provisional virtual viewpoint in the three-dimensional image of the endoscope using the radiation image and the three-dimensional image;
deriving a virtual viewpoint at the first time point in the three-dimensional image of the endoscope using the provisional virtual viewpoint, the first real endoscopic image, and the three-dimensional image; and
deriving a virtual viewpoint at a second time point after the first time point in the three-dimensional image of the endoscope using the first real endoscopic image and a second real endoscopic image captured by the endoscope at the second time point.
